(19) United States
(12) Patent Application Publication
(10) Pub. No.: US 2024/0425463 A1
(43) Pub. Date: Dec. 26, 2024

(54) PROCESS FOR MANUFACTURING A DIPHENYLPHRAZINE DERIVATIVE

(71) Applicant: ACTELION PHARMACEUTICALS LTD, Allschwil (CH)

(72) Inventors: Ronny VANIERSCHOT, Beerse (BE); Alain COLLAS, Beerse (BE); Carina LEYS, Beerse (BE); Kore VAN MECHELEN, Beerse (BE)

(21) Appl. No.: 18/262,777

(22) PCT Filed: Jan. 28, 2022
(86) PCT No.: PCT/EP2022/052084

§ 371 Date: Jul. 25, 2023

(30) Foreign Application Priority Data
Jan. 29, 2021 (WO) PCT/EP2021/052209
Nov. 24, 2021 (WO) PCT/EP2021/082836

(51) Int. Cl. C07D 241/20; A61K 31/4965

(52) U.S. Cl. CPC C07D 241/20; A61K 31/4965

(57) ABSTRACT

The present invention relates to a process for the manufacturing of calcium; {4-[(5, 6-diphenylpyrazin-2-yl)(propan-2-yl)amino]butoxy}acetate, or a pharmaceutically acceptable hydrate or solvate thereof. Moreover, it relates to calcium; {4-[(5,6-diphenylpyrazin-2-yl)(propan-2-yl)amino]butoxy}acetate with high purity, as well as to crystalline forms of calcium; {4-[(5,6-diphenylpyrazin-2-yl)(propan-2-yl)amino]butoxy}acetate and hydrates and solvates thereof. Furthermore, the invention relates to the use of calcium; {4-[(5, 6-diphenylpyrazin-2-yl)(propan-2-yl)amino]butoxy}acetate for the treatment or prevention of e.g. pulmonary arterial hypertension (PAH) or chronic thromboembolic pulmonary hypertension (CTEPH).

### FIELD OF THE INVENTION

The present invention relates to a process for the manufacturing of calcium; {4-[(5,6-diphenylpyrazin-2-yl)(propan-2-yl)amino]butoxy}acetate, or a pharmaceutically acceptable hydrate or solvate thereof:

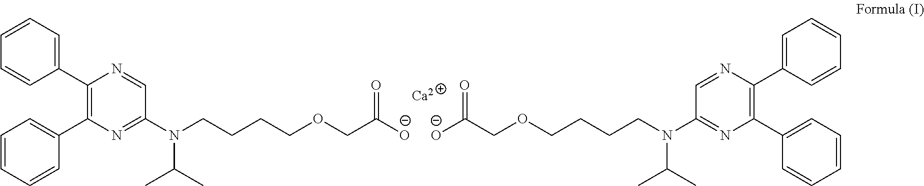 [SMILES: CC(C)N(CCCCOCC(=O)[O-])c1cnc(-c2ccccc2)c(-c2ccccc2)n1 ; CC(C)N(CCCCOCC(=O)[O-])c1cnc(-c2ccccc2)c(-c2ccccc2)n1]

The compound of formula (I) is the calcium salt of the metabolite of selexipag (calcium salt of ACT-333679), and has the formula Ca(C₂₅H₂₈N₃O₃)₂, i.e. C₅₀H₅₆N₆O₆Ca (MW: 877.109). In the present invention, the terms “calcium; {4-[(5,6-diphenylpyrazin-2-yl)(propan-2-yl)amino]butoxy}acetate”, “calcium; 2-[4-[(5,6-diphenylpyrazin-2-yl)(propan-2-yl)amino]butoxy]acetate”, “calcium; 2-[4-[(5,6-diphenylpyrazin-2-yl)-isopropylamino]butoxy]acetate”, “calcium; 2-[4-[(5,6-diphenylpyrazin-2-yl)-(propan-2-yl)-amino]butoxy]acetate”, “calcium salt of {4-[(5,6-diphenylpyrazin-2-yl)(propan-2-yl)amino]butoxy}acetic acid”; “calcium salt of {4-[(5,6-diphenylpyrazin-2-yl)(isopropyl)amino]butoxy}acetic acid”; “calcium salt of 2-(4-((5,6-diphenylpyrazin-2-yl)(propan-2-yl)amino)butoxy)acetic acid”; “calcium salt of 2-(4-((5,6-diphenylpyrazin-2-yl)(isopropyl)amino)butoxy)acetic acid”; “bis[[2-[4-[(5,6-diphenylpyrazin-2-yl)-isopropylamino]butoxy]acetyl]oxy]calcium)” and calcium salt of the metabolite of selexipag (calcium salt of ACT-333679) are used synonymously.

Selexipag (INN) is 2-{4-[(5,6-diphenylpyrazin-2-yl)(propan-2-yl)amino]butoxy}-N-(methanesulfonyl)acetamide (ACT-293987, NS-304, CAS: 475086-01-2; 2-{4-[N-(5,6-diphenylpyrazin-2-yl)-N-isopropylamino]butyloxy}-N-(methylsulfonyl)acetamide), also known as Uptravi™. The metabolite of selexipag is 2-(4-((5,6-diphenylpyrazin-2-yl)(isopropyl)amino)butoxy)acetic acid (MRE-269, ACT-333679, 2-{4-[(5,6-diphenylpyrazin-2-yl)-propan-2-ylamino]butoxy}acetic acid; {4-[(5,6-diphenylpyrazin-2-yl)(isopropyl)amino]butoxy}acetic acid; {4-[(5,6-diphenylpyrazin-2-yl)-(propan-2-yl)amino]butoxy}acetic acid; CAS: 475085-57-5 (MW 419.52)).

The present invention relates to a process for the manufacturing of calcium; {4-[(5,6-diphenylpyrazin-2-yl)(propan-2-yl)amino]butoxy}acetate, or a pharmaceutically acceptable hydrate or solvate thereof. Moreover, it relates to calcium; {4-[(5,6-diphenylpyrazin-2-yl)(propan-2-yl)amino]butoxy}acetate with high purity, as well as to crystalline forms of calcium; {4-[(5,6-diphenylpyrazin-2-yl)(propan-2-yl)amino]butoxy}acetate and hydrates and solvates thereof. Furthermore, the invention relates to the use of calcium; {4-[(5,6-diphenylpyrazin-2-yl)(propan-2-yl)amino]butoxy}acetate for the treatment or prevention of e.g. pulmonary arterial hypertension (PAH) or chronic thromboembolic pulmonary hypertension (CTEPH).

### BACKGROUND OF THE INVENTION

The preparation and the medicinal use of selexipag and its active metabolite 2-(4-((5,6-diphenylpyrazin-2-yl)(isopropyl)amino)butoxy)acetic acid is described in WO2002/088084; WO2009/157396; WO2009/107736; WO2009/154246; WO2009/157397; WO2009/157398; WO2010/150865; WO2011/024874; Nakamura et al., Bioorg Med Chem (2007), 15, 7720-7725; Kuwano et al., J Pharmacol Exp Ther (2007), 322(3), 1181-1188; Kuwano et al., J Pharmacol Exp Ther (2008), 326(3), 691-699; O. Sitbon et al., N Engl J Med (2015), 373, 2522-33; Asaki et al., Bioorg Med Chem (2007), 15, 6692-6704; Asaki et al., J. Med. Chem. (2015), 58, 7128-7137. Salts of selexipag metabolite are described in JP 2019-149945.

Selexipag was shown to be beneficial in the treatment of pulmonary arterial hypertension. In a phase Ill clinical trial, among patients with pulmonary arterial hypertension, the risk of the primary composite end point of death or a complication related to pulmonary arterial hypertension was significantly lower among patients who received selexipag than among those who received placebo. Selexipag received market approval e.g. in the US and is indicated for the treatment of pulmonary arterial hypertension (PAH, WHO Group I) to delay disease progression and reduce the risk of hospitalization for PAH.

So far, standard film-coated tablet formulations of selexipag intended for twice daily oral administration have been used, wherein excipients comprise D-mannitol, corn starch, low substituted hydroxypropylcellulose, hydroxypropylcellulose, and magnesium stearate; the tablets are film coated with a coating material containing hypromellose, propylene glycol, titanium dioxide, carnauba wax along with mixtures of iron oxides.

Moreover, a safety study of the switch from oral selexipag to intravenous selexipag in patients with PAH has been conducted (NCT03187678), whereby selexipag was administered twice daily as an infusion of approximately 87 min. The dose was individualized for each patient to correspond to his/her current oral dose of selexipag.

Selexipag is thought to function as a prodrug (while retaining some agonistic activity on the IP receptor on its own) which can exert long-lasting selective IP receptor agonist activity of the active metabolite 2-(4-((5,6-diphenylpyrazin-2-yl)(isopropyl)amino)butoxy)acetic acid in mammals, especially humans. The in vivo metabolism of selexipag effectively may act as a kind of ‘slow-release mechanism’ that potentially both prolongs activity and reduces typical adverse effects associated with high concentrations of PGI2 agonists (Kuwano et al., J Pharmacol Exp Ther (2007), 322(3), 1181-1188).

In certain instances, the use of an oral formulation of selexipag may be inappropriate or impossible, e.g. in urgent care, or in case a patient is for some reasons unable to swallow a tablet.

Moreover, in general, it is desirable to reduce the drug burden, particularly for treatment regimens that may last several months or more.

The number and/or volume of dosage forms that need to be administered are commonly referred to as “drug burden”. A high drug burden is undesirable for many reasons, such as the frequency of administration, often combined with the inconvenience of having to swallow large dosage forms, as well as the need to store and transport a large number or volume of pharmaceutical formulations. A high drug burden increases the risk of patients not taking their entire dose, thereby failing to comply with the prescribed dosage regimen.

Therefore, there is a need to develop a pharmaceutical composition or formulation, whose pharmaceutical effect is maintained, for example, for one week or longer, or one month or longer, whereby it only has to be administered at long time intervals such as one week or longer, or even one month or longer (a long-acting formulation), i.e. three months.

Calcium; {4-[(5,6-diphenylpyrazin-2-yl)(propan-2-yl)amino]butoxy}acetate, or a pharmaceutically acceptable hydrate or solvate thereof, which is produced according to the process of the present invention, is particularly suitable for long-acting formulations, due to its low solubility in aqueous media. The present process allows to obtain calcium; {4-[(5,6-diphenylpyrazin-2-yl)(propan-2-yl)amino]butoxy}acetate, or a pharmaceutically acceptable hydrate or solvate thereof, in a high purity.

### SUMMARY OF THE INVENTION

It is an object of the present invention to provide an improved process for the manufacture of calcium; {4-[(5,6-diphenylpyrazin-2-yl)(propan-2-yl)amino]butoxy}acetate, or a pharmaceutically acceptable hydrate or solvate thereof. Moreover, it is an object of the present invention to provide new crystal forms of calcium; {4-[(5,6-diphenylpyrazin-2-yl)(propan-2-yl)amino]butoxy}acetate.

It has now been found that calcium; {4-[(5,6-diphenylpyrazin-2-yl)(propan-2-yl)amino]butoxy}acetate can be efficiently produced by the present process in high purity, i.e. avoiding excess of residual Ca²⁺, i.e. calcium salt from the starting material, in particular Ca(OH)₂ in the final product. In addition, the new process ensures improved conversion of the starting material selexipag metabolite, i.e. a lower proportion of excess selexipag metabolite in the product. Moreover, new crystal forms and hydrates/solvates have been produced. The new process may use various calcium salts to provide high yield, high conversion to the calcium salt, and high purity of calcium; {4-[(5,6-diphenylpyrazin-2-yl)(propan-2-yl)amino]butoxy}acetate.

Due to the low solubility in water of the calcium salt of selexipag metabolite obtained by the present process, this product and various crystalline forms are suitable for the manufacturing of long-acting formulations, such as for instance long-acting injectables. The improved process of the present invention allows for the production of particularly pure products, which is important in the manufacturing of drug compounds.

### DESCRIPTION OF THE FIGURES

FIG. 1 shows the X-ray powder diffraction diagram of calcium; {4-[(5,6-diphenylpyrazin-2-yl)(propan-2-yl)amino]butoxy}acetate in crystalline Form 1. The X-ray diffraction shows the following peaks: 5.1° (85%), 5.4° (20%), 8.8° (63%), 9.9° (100%), 11.4° (38%), 13.4° (21%), 13.8° (21%), 16.3° (65%), 18.1° (19%), 18.7° (27%), 19.7° (52%), 20.9° (51%), 21.4° (31%), 22.9° (51%), 23.6° (36%), 25.1° (37%).

The above-listed peaks describe the experimental results of the X-ray powder diffraction diagram shown in FIG. 1. It is understood that not all of these peaks are required to fully and unambiguously characterise Form 1.

FIG. 2 shows the X-ray powder diffraction diagram of calcium; {4-[(5,6-diphenylpyrazin-2-yl)(propan-2-yl)amino]butoxy}acetate in crystalline Form 2 as obtained from Example 5. The X-ray diffraction shows the following peaks: 3.2° (100%), 6.3° (21%), 7.7° (21%), 9.30 (34%), 10.0° (35%), 10.4° (9%), 11.6° (5%), 12.7° (26%), 13.8° (7%), 15.7° (12%), 17.5° (8%), 19.2° (20%), 20.2° (12%), 21.3° (8%), 22.9° (17%), 23.4° (13%), 24.0° (14%), 25.2° 2theta (6%).

The above-listed peaks describe the experimental results of the X-ray powder diffraction shown in FIG. 2. It is understood that not all of these peaks are required to fully and unambiguously characterise Form 2.

FIG. 3 shows the DSC curve of Form 2

FIG. 4 shows the TGA curve of Form 2

FIG. 5 shows the X-ray powder diffraction diagram of calcium; {4-[(5,6-diphenylpyrazin-2-yl)(propan-2-yl)amino]butoxy}acetate in crystalline Form 3 as obtained from Example 6. The X-ray diffraction shows the following peaks: 4.5° (100%), 4.8° (60%), 5.0° (56%), 7.9° (36%), 8.8° (47%), 9.0° (53%), 10.0° (74%), 11.9° (46%), 14.9° (50%), 15.6° (69%), 17.1° (43%), 18.7° (100%), 19.7° (33%), 20.7° (30%), 21.1° (17%), 22.1° (38%), 22.7° (34%), 23.9° (22%), 24.5° (12%), 26.1° 2theta (12%).

The above-listed peaks describe the experimental results of the X-ray powder diffraction diagram shown in FIG. 5. It is understood that not all of these peaks are required to fully and unambiguously characterise Form 3.

FIG. 6 shows the DSC curve of Form 3

FIG. 7 shows the TGA curve of Form 3

FIG. 8 shows the X-ray powder diffraction diagram of calcium; {4-[(5,6-diphenylpyrazin-2-yl)(propan-2-yl)amino]butoxy}acetate in crystalline Form 5 as obtained from Example 7. The X-ray diffraction shows the following peaks: 4.9° (25%), 8.8° (49%), 9.8° (100%), 11.0° (44%), 12.8° (21%), 13.1° (23%), 13.3° (17%), 14.7° (12%), 15.7° (17%), 16.1° (8%), 16.7° (17%), 16.9° (29%), 17.8° (5%), 18.2° (4%), 18.7° (10%), 19.0° (8%), 19.5° (43%), 20.1° (11%), 20.6° (10%), 21.1° (38%), 21.5° (22%), 22.6° (20%), 23.6° (12%), 26.3° (10%), 30.3° 2theta (7%).

The above-listed peaks describe the experimental results of the X-ray powder diffraction diagram shown in FIG. 8. It is understood that not all of these peaks are required to fully and unambiguously characterise Form 5.

In the X-ray diffraction diagrams of FIGS. 1, 2, 5 and 8 the angle of refraction 2theta (2θ) is plotted on the horizontal axis and the counts on the vertical axis.

FIG. 9 shows the plasma concentrations of the different studied formulations containing selexipag, selexipag metabolite (2-(4-((5,6-diphenylpyrazin-2-yl)(isopropyl)amino)-butoxy)acetic acid; ACT333679), and the calcium salt of selexipag metabolite (calcium; {4-[(5,6-diphenylpyrazin-2-yl)(propan-2-yl)amino]butoxy}acetate; Ca-salt of ACT333679) in function of time.

### DETAILED DESCRIPTION OF THE INVENTION

The present invention describes a process for the manufacturing of calcium; {4-[(5,6-diphenylpyrazin-2-yl)(propan-2-yl)amino]butoxy}acetate of formula (I), or a pharmaceutically acceptable hydrate or solvate thereof:

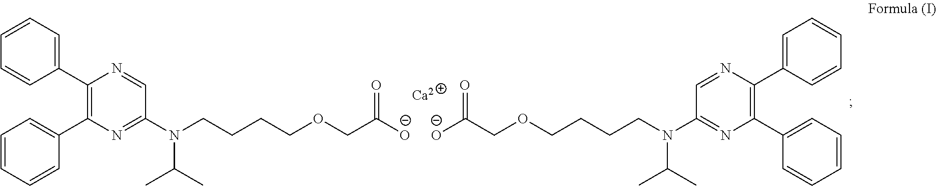 [SMILES: CC(C)N(CCCCOCC(=O)[O-])c1cnc(-c2ccccc2)c(-c2ccccc2)n1 ; CC(C)N(CCCCOCC(=O)[O-])c1cnc(-c2ccccc2)c(-c2ccccc2)n1]

comprising the steps of
mixing {4-[(5,6-diphenylpyrazin-2-yl)(propan-2-yl)amino]butoxy}acetic acid and a first calcium source with a solvent (a) to obtain a mixture;
heating or maintaining the mixture at a temperature in the range of 20° C. to 85° C.;
isolating the obtained solid product;
optionally re-slurrying the isolated solid product in a solution of a second calcium source in solvent (b) at a temperature in the range of 20° C. to 85° C.

In some embodiments, the mixing step comprises mixing {4-[(5,6-diphenylpyrazin-2-yl)(propan-2-yl)amino]butoxy}acetic acid and solvent (a) to obtain a mixture; and heating or maintaining the mixture at a temperature in the range of 20° C. to 85° C. prior to the addition of the first calcium source.

In some embodiments,
the first calcium source is dissolved in solvent (b) to obtain solution (b) prior to the addition of solution (b) to the mixture of {4-[(5,6-diphenylpyrazin-2-yl)(propan-2-yl)amino]butoxy}acetic acid and solvent (a).

The present invention is concerned with a process for the manufacturing of calcium; {4-[(5,6-diphenylpyrazin-2-yl)(propan-2-yl)amino]butoxy}acetate of formula (I), or a pharmaceutically acceptable hydrate or solvate thereof:

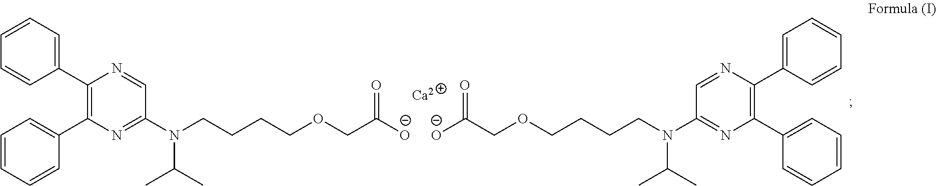 [SMILES: CC(C)N(CCCCOCC(=O)[O-])c1cnc(-c2ccccc2)c(-c2ccccc2)n1 ; CC(C)N(CCCCOCC(=O)[O-])c1cnc(-c2ccccc2)c(-c2ccccc2)n1]

comprising the steps of
(a) dissolving {4-[(5,6-diphenylpyrazin-2-yl)(propan-2-yl)amino]butoxy}acetic acid in a solvent (a) to obtain solution (a);
(b) heating solution (a) to a temperature in the range of 20° C. to 85° C.;
(c) dissolving a first calcium source in solvent (b) to obtain solution (b);
(d) dosing solution (b) to solution (a);
(e) isolating the obtained solid product;
(f) optionally re-slurrying the product of step (e) in a solution of a second calcium source in solvent (b) at a temperature in the range of 20° C. to 85° C.

In some embodiments, the first calcium source and the optional second calcium source is Ca(OAc)₂.

The starting material {4-[(5,6-diphenylpyrazin-2-yl)(propan-2-yl)amino]butoxy}acetic acid, i.e. selexipag metabolite (MRE-269, ACT-333679) can be prepared as known from the art, e.g. as described in EP1400518A1, example 42.

Calcium; {4-[(5,6-diphenylpyrazin-2-yl)(propan-2-yl)amino]butoxy}acetate, having the structure of formula (I) as indicated above, may be in anhydrous form, or in a hydrate form or a pharmaceutically acceptable solvate form. The term “pharmaceutically acceptable solvents” refers to solvents that retain the desired biological activity of the compound and exhibit minimal undesired toxicological effects. Preferred is an anhydrous form or a hydrate form.

Calcium; {4-[(5,6-diphenylpyrazin-2-yl)(propan-2-yl)amino]butoxy}acetate may be in a hydrate form. The hydrate form may be from about 0.1 to about 1 water molecules per calcium; {4-[(5,6-diphenylpyrazin-2-yl)(propan-2-yl)amino]butoxy}acetate molecules. In some embodiments, the molar ratio of water to calcium; {4-[(5,6-diphenylpyrazin-2-yl)(propan-2-yl)amino]butoxy}acetate ranges from about 0.1 to about 1, such as about 0.1 to about 0.15, about 0.15 to about 0.2, about 0.2 to about 0.25, about 0.25, to about 0.3, about 0.3 to about 0.35, about 0.35 to about 0.4, about 0.4 to about 0.45, about 0.45 to about 0.5, about 0.5 to about 0.55, about 0.55 to about 0.6, about 0.6 to about 0.65, about 0.65 to about 0.7, about 0.7 to about 0.75, about 0.75 to about 0.8, about 0.8 to about 0.85, about 0.85 to about 0.9, about 0.9 to about 0.95, about 0.95 to about 1. The molar ratio of water in the hydrate form may change based on storage conditions of the compound, the method of formation of the compound, and the crystal structure of the compound.

The solvent (a) for dissolving {4-[(5,6-diphenylpyrazin-2-yl)(propan-2-yl)amino]butoxy}-acetic acid in step (a) may be an organic solvent or a mixture of one or more organic solvent(s) with water. Solution (a) relates to a solution of {4-[(5,6-diphenylpyrazin-2-yl)(propan-2-yl)amino]butoxy}-acetic acid in solvent (a).

The water used in the present process preferably is purified water, e.g. standard purified water (PW).

Preferably, the organic solvent is miscible with water or partly soluble in water. Miscible with water in this context means miscibility, or solubility of at least 200 g/L water. Preferably, the organic solvent is miscible with water. Suitable organic solvents may be selected from the group consisting of acetone, tetrahydrofuran (THF), acetonitrile, MEK (methyl ethyl ketone), DMSO, DMF, 1,4-dioxane, pyridine, dimethylacetamide (DMA), methyl acetate (MeOAc), methanol, ethanol, propanol (1-propanol, 2-propanol), and butanol (1-butanol, 2-butanol, 2-methylpropan-1-ol, 2-methylpropanol). In one embodiment, the organic solvent may be chosen from the group consisting of acetone, THF, acetonitrile, MEK (methyl ethyl ketone), DMSO, DMF, 1,4-dioxane, pyridine, dimethylacetamide (DMA), methyl acetate (MeOAc), propanol and butanol, or a mixture thereof. Preferred organic solvents are acetone and THF, in particular acetone.

The organic solvent in solvent (a) may be mixed with water. The ratio is given in % w/w. Hence, the ratio of solvent (a)/water (w/w) may be from 100/0 to 10/90, or from 100/0 to 50/50, or from 100/0 to 70/30. For instance, solvent (a) is a mixture of acetone/water in a ratio from 100/0 to 30/70, or a mixture of THF/water in a ratio from 100/0 to 10/90.

Solvent (a) may be for instance acetone/water in a ratio from 100/0 to 80/20, or from 99/1 to 90/10, for instance 95/5.

The concentration of starting material {4-[(5,6-diphenylpyrazin-2-yl)(propan-2-yl)amino]butoxy}acetic acid in solvent (a) is not particularly limited. The concentration may be selected from a range of 60 g {4-[(5,6-diphenylpyrazin-2-yl)(propan-2-yl)amino]butoxy}acetic acid per 100 g solvent (a) or lower, for instance from 1 g/100 g solvent (a) to 60 g/100 g solvent (a), or from 1 g/100 g solvent (a) to 50 g/100 g solvent (a), or from 1 g/100 g solvent (a) to 40 g/100 g solvent (a). For example, the concentration may be from 1 g selexipag metabolite/100 g acetone/water (95/5) to 10.2 g selexipag metabolite/100 g acetone/water 95/5, for instance 8 to 9 g±10% or 8 to 9 g±5% selexipag metabolite/100 g acetone/water 95/5.

In step (b), solution (a) is heated to a temperature ranging from 20° C. to 85° C. The temperature depends on the boiling point of solvent (a), and is selected high enough for dissolving the starting material, and low enough to prevent degradation of the starting material. In some embodiments, the mixture of {4-[(5,6-diphenylpyrazin-2-yl)(propan-2-yl)amino]butoxy}acetic acid and solvent (a) is heated or maintained at a temperature in the range of 20° C. to 85° C. In some embodiments, the mixture of {4-[(5,6-diphenylpyrazin-2-yl)(propan-2-yl)amino]butoxy}acetic acid, the first calcium source, and solvent (a) is heated or maintained at a temperature in the range of 20° C. to 85° C.

In one embodiment, the temperature of step (b) or the mixture ranges from 20° C. to 85° C., from 20° C. to 80° C., from 20° C. to 75° C., from 20° C. to 70° C., from 20° C. to 65° C., from 20° C. to 60° C., from 20° C. to 55° C., for instance from 20° C. to 50° C.±3° C. Preferably, the end-temperature in step (b) or the mixture is higher than 20° C., and is ranging from 40° C. to 85° C., from 45° C. to 80° C., from 45° C. to 75° C., from 45° C. to 70° C., from 45° C. to 65° C., from 45° C. to 60° C., from 45° C. to 55° C., for instance 50° C.±3° C.

Preferably, the end-temperature in step (b) or the mixture is reached by heating swiftly, for instance at 1 K/min. Alternatively, the starting material could be added to solvent (a) set at the desired temperature.

Optionally, solution (a) or the mixture of {4-[(5,6-diphenylpyrazin-2-yl)(propan-2-yl)amino]butoxy}acetic acid and solvent (a) maybe subjected to a filter step. Preferably, the optional filtering step is a polish filtering step. The mesh size of the filter may be 5 μm or lower, for instance ranging from 0.2 μm-5 μm, for example 0.5 μm.

Optionally, seed crystals of calcium; {4-[(5,6-diphenylpyrazin-2-yl)(propan-2-yl)amino]butoxy}acetate may be added to solution (a) or the mixture. The addition of seed crystals is not mandatory, i.e. the process works without adding seed crystals, and affords the same crystal form than without seeding. However, seed crystals may be added in order to optimize the crystallisation process. The purity of the product is not influenced by the addition of seed crystals.

Seed crystals may optionally be added, whereby the amount of seed crystals is not particularly limited. However, for economic reasons, amounts of seed crystals may be selected in an amount of up to 25% w/w in respect of amount of starting material {4-[(5,6-diphenylpyrazin-2-yl)(propan-2-yl)amino]butoxy}acetic acid; or in an amount of 0% w/w to 25% w/w in respect of amount of starting material {4-[(5,6-diphenylpyrazin-2-yl)(propan-2-yl)amino]butoxy}acetic acid. In one embodiment, seed crystals are added in an amount of 0.5% w/w to 10% w/w, or in an amount of 0.5% w/w to 5% w/w, or in an amount of 0.5% w/w to 4% w/w, or in an amount of 0.5% w/w to 3% w/w, for instance in an amount of 1% w/w±10%, or 1% w/w±5%.

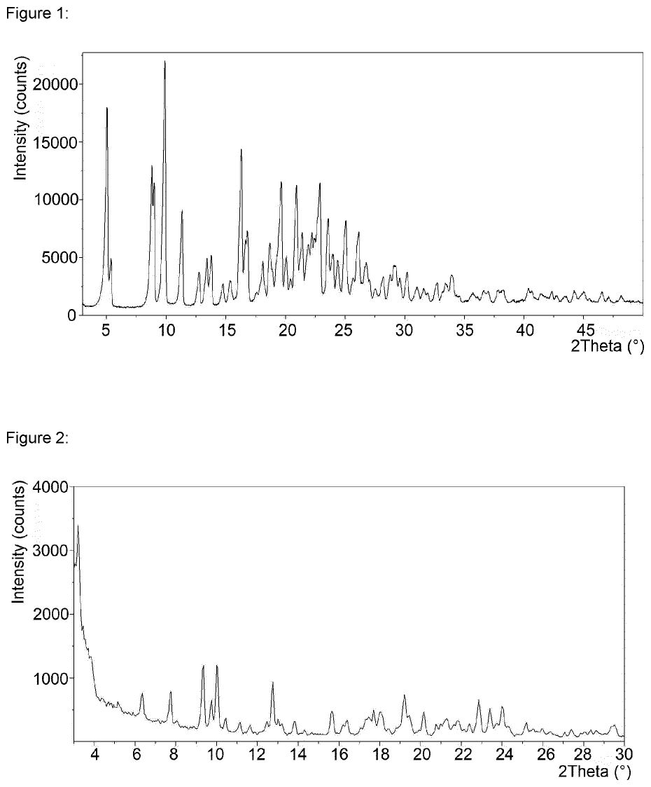

In one embodiment, seed crystals have an X-ray powder diffraction pattern with at least five peaks, or at least seven peaks, or at least nine peaks having angle of refraction 29 (2theta) values selected from: 5.1°, 5.4°, 8.8°, 99°, 11.4°, 13.4°, 13.8°, 16.3°, 19.7°, 20.9°, 21.4°, 22.9°, 25.1°. In one embodiment, seed crystals have an X-ray powder diffraction pattern with at least five peaks, or at least seven peaks, or at least nine peaks having angle of refraction 29 (2theta) values selected from: 5.1°, 5.4°, 8.8°, 99°, 11.4°, 13.4°, 13.8°, 16.3°, 18.1°, 18.7°, 19.7°, 20.9°, 21.4°, 22.9°, 23.6°, 25.1°. Specifically, crystalline Form 1 shows an X-ray powder diffraction diagram with the following peaks and their relative intensity given in parenthesis: 5.1° (85%), 5.4° (20%), 8.8° (63%), 9.9° (100%), 11.4° (38%), 13.4° (21%), 13.8° (21%), 16.3° (65%), 18.1° (19%), 18.7° (27%), 19.7° (52%), 20.9° (51%), 21.4° (31%), 22.9° (51%), 23.6° (36%), 25.1° (37%), wherein said X-ray powder diffraction diagram is obtained by using combined Cu Kα1 and Kα2 (Kalpha2) radiation, without Kα2 stripping; and the accuracy of the 2θ (2theta) values is in the range of 2θ+/−0.2° (2theta+/−0.2°). Most preferably, the seed crystals show the X-ray powder diffraction pattern as depicted in FIG. 1. This crystalline form is indicated herein as Form 1.

One skilled in the art is aware of the fact that relative peak intensities will show inter-apparatus variability as well as variability due to degree of crystallinity, preferred orientation, prepared sample surface, and other factors known to those skilled in the art, and should be taken as a qualitative measure only. One of ordinary skill in the art will also understand that an X-ray diffraction pattern may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles.

Optionally, a waiting step may follow the addition of the seed crystals.

In some embodiments, the first calcium source is dissolved in solvent (b) before being added to solution (a). Dissolving of starting material selexipag metabolite in solvent (a) and the calcium source in solvent (b) can be performed in parallel. Solution (b) relates to a solution of the calcium source in solvent (b).

The first calcium source provides Ca²⁺ which may be dissolved in solvent (b). In some embodiments, the first calcium source is selected from Ca(OAc)₂, calcium propionate, calcium formate, and calcium pantothenate. In some embodiments, the first calcium source is selected from Ca(OAc)₂, calcium propionate, and calcium formate. In some embodiments, the first calcium source is Ca(OAc)₂. In some embodiments, the first calcium source is calcium propionate. In some embodiments, the first calcium source is calcium formate. In some embodiments, the first calcium source is calcium pantothenate. Solvent (b) may be selected from water or a mixture of water and an organic solvent. The organic solvent may be one of the organic solvents listed above, or a mixture thereof. Thereby, the proportion of water admixed with the organic solvent is higher, i.e. the ratio of water/organic solvent (w/w) is from 100/0 to 50/50, or from 100/0 to 55/45, or from 100/0 to 70/30, or from 100/0 to 75/25, or from 100/0 to 80/20, or from 100/0 to 90/10, or from 100/0 to 95/5. Preferably, solvent (b) is water.

The concentration of the first calcium source in solvent (b) is not particularly limited. In one embodiment, the concentration of the first calcium source in solution (b) is the saturation concentration or less, for instance in a range from 0.5 g of the first calcium source per 100 g solvent (b) to 35 g of the first calcium source per 100 g solvent (b). In one embodiment, the concentration of solution (b) is 35 g of the first calcium source per 100 g solvent (b) or less, for instance in a range from 1 g of the first calcium source per 100 g solvent (b) to 35 g of the first calcium source per 100 g solvent (b); or from 1 g of the first calcium source per 100 g solvent (b) to 30 g of the first calcium source per 100 g solvent (b); or from 1 g of the first calcium source per 100 g solvent (b) to 25 g of the first calcium source per 100 g solvent (b); or from 1 g of the first calcium source per 100 g solvent (b) to 20 g of the first calcium source per 100 g solvent (b); or from 1 g of the first calcium source per 100 g solvent (b) to 15 g of the first calcium source per 100 g solvent (b). For instance, 35 g of the first calcium source per 100 g water or less, for instance in a range from 1 g of the first calcium source per 100 g water to 35 g of the first calcium source per 100 g water; or from 1 g of the first calcium source per 100 g water to 30 g of the first calcium source per 100 g water; or from 1 g of the first calcium source per 100 g water to 25 g of the first calcium source per 100 g water; or from 1 g of the first calcium source per 100 g water to 20 g of the first calcium source per 100 g water; or from 1 g of the first calcium source per 100 g water to 15 g of the first calcium source per 100 g water. For instance, 5 g of the first calcium source per 100 g water to 15 g of the first calcium source per 100 g water; or 7 g of the first calcium source per 100 g water to 13 g of the first calcium source per 100 g water; or 9 g±5% of the first calcium source per 100 g water to 10 g±5% of the first calcium source per 100 g water

The amount of the first calcium source added in step (c) and (d) or to the {4-[(5,6-diphenylpyrazin-2-yl)(propan-2-yl)amino]butoxy}acetic acid is 0.4 mol to 1 mol per mol {4-[(5,6-diphenylpyrazin-2-yl)(propan-2-yl)amino]butoxy}acetic acid (starting material selexipag metabolite), or from 0.4 to 0.8 mol per mol starting material, or from 0.4 to 0.6 mol per mol starting material, or from 0.45 to 0.6 mol per mol starting material, or from 0.45 to 0.55 mol per mol starting material or from 0.5 to 0.6 mol per mol starting material, or from 0.5 to 0.55 mol per mol starting material, for instance 0.525±5% mol per mol starting material.

Optionally, step (d) or the step of adding the first calcium source may be divided into two or more dosage steps. This means that the amount of the first calcium source is added in one or more dosages. There may be a waiting step between the addition of the preceding and the subsequent first calcium source dosage.

For instance, in a first dosage step, the amount of the first calcium source may comprise 5 to 50% of the total amount of the first calcium source, or 5 to 40% of the total amount of the first calcium source, or 5 to 35% of the total amount of the first calcium source, or 5 to 30% of the total amount of the first calcium source, or 5 to 25% of the total amount of the first calcium source, or 10 to 20% of the total amount of the first calcium source, for example about 15% of the total amount of the first calcium source (to be understood as dissolved in solvent (b), “about” means±10% of 15%).

Optionally, a waiting or aging step may follow to a dosage step. For instance, a waiting or aging step of 1 to 48 h may follow a first dosage of 5 to 50% of the total amount of the first calcium source. The duration time of the waiting step depends on the scale of the preparation batch, and may be even longer. The waiting or aging step may range for example from 1 to 48 h, from 1 to 24 h, from 1 to 15 h, from 1 to 12 h, or from 1 to 10 h.

In case only a part of the total amount of the first calcium source is added in a first dosage step, the remaining amount of the first calcium source may be added in a second, or subsequent dosage step. Preferably, the remaining amount of the first calcium source is added in a second dosage step.

The dosing of the first calcium source is preferably linear controlled in each dosage step.

Preferably, the mixture obtained in step (d) or the mixture of {4-[(5,6-diphenylpyrazin-2-yl)(propan-2-yl)amino]butoxy}acetic acid, the first calcium source and solvent (a) is further stirred for at least 30 minutes, for instance for 30 min to 48 h, for 30 min to 24 h, for 30 min to 12 h, or for 30 min to 10 h.

Afterwards, the obtained solid product is isolated in step (e), for instance by filtration or centrifugation.

Optionally, the product obtained in step (e) or the isolated solid product is washed with a solvent (c), preferably with a mixture of water and an organic solvent, wherein the organic solvent is selected from the organic solvents as described above. The ratio is given in % w/w. Hence, the ratio of solvent (a)/water (w/w) may be from 100/0 to 10/90, or from 100/0 to 50/50, or from 100/0 to 70/30. For instance, solvent (a) is a mixture of acetone/water in a ratio from 100/0 to 50/50, or a mixture of THF/water in a ratio from 100/0 to 10/90.

Each of the steps (d) to (e) or the addition of the first calcium source and the isolating step, as well as the optional filtration and waiting/stirring steps, are performed at a temperature form 20° C. to 85° C., for instance the temperature selected in step (b), for example at the end-temperature of step (b) or at a lower temperature.

The obtained product may then be subjected to a drying step, preferably under vacuum and nitrogen purge. Preferably, the drying temperature is from 20° C. to 85° C., or from 25° C. to 85° C., for instance from 30° C. to 80° C., or from 40° C. to 55° C., or from 45° C. to 55° C., for instance at 50° C.±3° C.

Optionally, the process for the production of calcium; {4-[(5,6-diphenylpyrazin-2-yl)(propan-2-yl)amino]butoxy}acetate of formula (I) may comprise a re-slurrying step (f). Such re-slurrying step is suitable in case the product of step (e) contains an excess of starting material {4-[(5,6-diphenylpyrazin-2-yl)(propan-2-yl)amino]butoxy}acetic acid (i.e. free {4-[(5,6-diphenylpyrazin-2-yl)(propan-2-yl)amino]butoxy}acetic acid), for instance more than 2% of starting material {4-[(5,6-diphenylpyrazin-2-yl)(propan-2-yl)amino]butoxy}acetic acid.

In such case, the product of step (e) or the isolated solid product may be re-slurried in a solution of the second calcium source in solvent (b), preferably of the second calcium source in water, at a temperature in the range from 20° C. to 85° C.

The second calcium source provides Ca²⁺ which may be dissolved in solvent (b). In some embodiments, the second calcium source is selected from Ca(OAc)₂, calcium propionate, calcium formate, and calcium pantothenate. In some embodiments, the second calcium source is selected from Ca(OAc)₂, calcium propionate, calcium formate. In some embodiments, the second calcium source is Ca(OAc)₂. In some embodiments, the second calcium source is calcium propionate. In some embodiments, the second calcium source is calcium formate. In some embodiments, the second calcium source is calcium pantothenate.

The general conditions of the re-slurrying step are comparable to those of steps (c) to (e), though not exactly the same conditions of the preceding steps need to be chosen, but can vary in the general ranges as given above. This means that the temperature is preferably the same as in step (b), for instance the end-temperature of step (b). Moreover, the solvent and concentration of the second calcium source is preferably the same as in step (c), for instance the solvent is water.

The product obtained in step (f) is isolated in the same way as in step (e), preferably washed with purified water and at drying conditions identical to the isolation of the primary crystallisation.

The calcium; {4-[(5,6-diphenylpyrazin-2-yl)(propan-2-yl)amino]butoxy}acetate of formula (I) as obtained by the present process is characterized by a high purity, in particular in respect of excess of Ca²⁺ stemming from the production process, i.e. from the inorganic Ca salt used as starting material. For instance, a process using Ca(OH)₂ as starting material produces an excess of residual Ca(OH)₂ which remains in the product. This is avoided by the present process. The calcium; {4-[(5,6-diphenylpyrazin-2-yl)(propan-2-yl)amino]butoxy}acetate of formula (I) obtainable by the present process is characterized by a Ca²⁺ content of 7.0±0.1% w/w or less, or by a Ca²⁺ content of 6.0±0.1% w/w or less, or by a Ca²⁺ content of 5.5±0.1% w/w or less, or by a Ca²⁺ content of 5.0±0.1% w/w or less. For instance, a Ca²⁺ content of 7.0±0.1% w/w to 4.0±0.1% w/w, or from 7.0±0.1% w/w to 4.1±0.1% w/w, or from 6.0±0.1% w/w to 4.0±0.1% w/w, or from 6.0±0.1% w/w to 4.1±0.1% w/w, or from 5.5±0.1% w/w to 4.0±0.1% w/w, or from 5.5±0.1% w/w to 4.1±0.1% w/w, or from 5.0±0.1% w/w to 4.0±0.1% w/w, or from 5.5±0.1% w/w to 4.1±0.1% w/w, or from 5.0±0.1% w/w to 4.1±0.1% w/w. The Ca²⁺ content is measured by ion chromatography, which is well known in the art. A suitable measurement method is exemplified in the experimental part.

Hence, the present invention also relates to a product obtainable by the process as described herein.

The product obtainable by the present process is particularly suitable for the manufacturing of long-acting formulations. This is shown in example 8, indicating the feasibility of long-acting formulations of calcium; {4-[(5,6-diphenylpyrazin-2-yl)(propan-2-yl)amino]butoxy}acetate in comparison to selexipag and selexipag metabolite {4-[(5,6-diphenylpyrazin-2-yl)(propan-2-yl)amino]butoxy}acetic acid.

Moreover, the following crystalline forms of calcium; {4-[(5,6-diphenylpyrazin-2-yl)(propan-2-yl)amino]butoxy}acetate are disclosed:
(i) Crystalline Form 2 of calcium; {4-[(5,6-diphenylpyrazin-2-yl)(propan-2-yl)amino]butoxy}-acetate of formula (I) has an X-ray powder diffraction pattern with at least five peaks having angle of refraction 2θ (2theta) values selected from 3.2°, 6.3°, 7.7°, 9.3°, 10.4°, 11.6°, 24.0° 2theta; preferably at least five peaks, or at least seven peaks, or at least nine peaks selected from 3.2°, 6.3°, 7.7°, 9.3°, 10.0°, 10.4°, 11.6°, 12.7°, 19.2°, 22.9°, 24.0° 2theta; especially at least five peaks, or at least seven peaks, or at least nine peaks selected from 3.2°, 6.3°, 77°, 93°, 10.0°, 10.4°, 11.6°, 12.7°, 13.8°, 15.7°, 17.5°, 19.2°, 20.2°, 21.3°, 22.9°, 23.4°, 24.0°, 25.2° 2theta,
   wherein said X-ray powder diffraction diagram is obtained by using Cu Kα1 radiation (with Kα2 stripping); and the accuracy of the 2θ (2theta) values is in the range of 2θ+/−0.2° (2theta+/−0.2°).
   Specifically, crystalline Form 2 shows an X-ray powder diffraction diagram with the following peaks and their relative intensity given in parenthesis: 3.2° (100%), 6.3° (21%), 7.7° (21%), 9.3° (34%), 10.0° (35%), 10.4° (9%), 11.6° (5%), 12.7° (26%), 13.8° (7%), 15.7° (12%), 17.5° (8%), 19.2° (20%), 20.2° (12%), 21.3° (8%), 22.9° (17%), 23.4° (13%), 24.0° (14%), 25.2° 2theta (6%).
(ii) Crystalline Form 3 of calcium; {4-[(5,6-diphenylpyrazin-2-yl)(propan-2-yl)amino]butoxy}-acetate of formula (I) has an X-ray powder diffraction pattern with peaks having angle of refraction 2θ (2theta) values selected from 4.5°, 7.9°, or 11.9°2theta; preferably at least five peaks, or at least seven peaks, or at least nine peaks selected from 4.5°, 4.8°, 5.0°, 7.9°, 10.0°, 11.9°, 14.9°, 15.6°, 17.1°, 18.7°, 22.1° and 22.7° 2theta; especially at least five peaks, or at least seven peaks, or at least nine peaks selected from 4.5°, 4.8°, 5.0°, 79°, 8.8°, 9.0°, 10.0°, 11.9°, 14.9°, 15.6°, 17.1°, 18.7°, 19.7°, 20.7°, 21.1°, 22.1°, 22.7°, 23.9°, 24.5°, 26.1° 2theta,
   wherein said X-ray powder diffraction diagram is obtained by using combined Cu Kα1 and Kα2 (Kalpha2) radiation, without Kα2 stripping; and the accuracy of the 2θ (2theta) values is in the range of 2θ+/−0.2° (2theta+/−0.2°).
   Specifically, crystalline Form 3 shows an X-ray powder diffraction diagram with the following peaks and their relative intensity given in parenthesis: 4.5° (100%), 4.8° (60%), 5.0° (56%), 7.9° (36%), 8.8° (47%), 9.0° (53%), 10.0° (74%), 11.9° (46%), 14.9° (50%), 15.6° (69%), 17.1° (43%), 18.7° (100%), 19.7° (33%), 20.7° (30%), 21.1° (17%), 22.1° (38%), 22.7° (34%), 23.9° (22%), 24.5° (12%), 26.1° 2theta (12%).
   Crystalline Form 3 is an Isostructural Solvate.
(iii) Crystalline Form 5 of calcium; {4-[(5,6-diphenylpyrazin-2-yl)(propan-2-yl)amino]butoxy}-acetate of formula (I) has an X-ray powder diffraction pattern with at least five peaks, or at least seven peaks, or at least nine peaks having angle of refraction 2θ (2theta) values selected from 4.9°, 8.8°, 9.8°, 11.0°, 12.8°, 13.1°, 16.9°, 19.5°, 21.1°, 21.5° and 22.6° 2theta; especially at least five peaks, or at least seven peaks, or at least nine peaks having angle of refraction 2θ (2theta) values selected from 4.9°, 8.8°, 9.8°, 11.0°, 12.8°, 13.1°, 13.3°, 14.7°, 15.7°, 16.1°, 16.7°, 16.9°, 17.8°, 18.2°, 18.7°, 19.0°, 19.5°, 20.1°, 20.6°, 21.1°, 21.5°, 22.6°, 23.6°, 26.3°, 30.3° 2theta,
   wherein said X-ray powder diffraction diagram is obtained by using Cu Kα1 radiation (with Kα2 stripping); and the accuracy of the 2θ (2theta) values is in the range of 2θ+/−0.2° (2theta+/−0.2°).
   Specifically, crystalline Form 5 shows an X-ray powder diffraction diagram with the following peaks and their relative intensity given in parenthesis: 4.9° (25%), 8.8° (49%), 9.8° (100%), 11.0° (44%), 12.8° (21%), 13.1° (23%), 13.3° (17%), 14.7° (12%), 15.7° (17%), 16.1° (8%), 16.7° (17%), 16.9° (29%), 17.8° (5%), 18.2° (4%), 18.7° (10%), 19.0° (8%), 19.5° (43%), 20.1° (11%), 20.6° (10%), 21.1° (38%), 21.5° (22%), 22.6° (20%), 23.6° (12%), 26.3° (10%), 30.3° 2theta (7%).

It is understood, that the crystalline forms of calcium; {4-[(5,6-diphenylpyrazin-2-yl)(propan-2-yl)amino]butoxy}-acetate of formula (I) may comprise non-coordinated and/or coordinated solvent. Coordinated solvent is used herein as term for a crystalline solvate. Likewise, non-coordinated solvent is used herein as term for physiosorbed or physically entrapped solvent (definitions according to Polymorphism in the Pharmaceutical Industry (Ed. R. Hilfiker, V C H, 2006), Chapter 8: U. J. Griesser: The Importance of Solvates).

Crystalline Form 1 in particular is a hydrate. It contains about 0.25 eq H₂O (about means±10%, for example ±5%).

Crystalline Form 2 in particular is an anhydrate, i.e. it comprises no coordinated water, but may comprise non-coordinated methanol.

Crystalline Form 3 in particular is an isostructural solvate, i.e. it comprises coordinated anisole or toluene.

Crystalline for Form 5 in particular is an anhydrate.

Moreover, one embodiment relates to a pharmaceutical composition comprising calcium; {4-[(5,6-diphenylpyrazin-2-yl)(propan-2-yl)amino]butoxy}-acetate of crystalline Form 2, crystalline Form 3 or crystalline Form 5 as described herein.

One embodiment relates to calcium; {4-[(5,6-diphenylpyrazin-2-yl)(propan-2-yl)amino]butoxy}-acetate of formula (I) as prepared by the process described herein for use in the treatment and/or prevention of a disease and/or disorder selected from the group consisting of ulcer, digital ulcer, diabetic gangrene, diabetic foot ulcer, pressure ulcer (bedsore), hypertension, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud's disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved [e.g., renal diseases such as tubulointerstitial nephritis), respiratory diseases (e.g., interstitial pneumonia, (idiopathic) pulmonary fibrosis, chronic obstructive pulmonary disease), digestive diseases (e.g, hepatocirrhosis, viral hepatitis, chronic pancreatitis and scirrhous stomachic cancer), cardiovascular diseases (e.g, myocardial fibrosis), bone and articular diseases (e.g, bone marrow fibrosis and rheumatoid arthritis), skin diseases (e.g, cicatrix after operation, scalded cicatrix, keloid, and hypertrophic cicatrix), obstetric diseases (e.g., hysteromyoma), urinary diseases (e.g., prostatic hypertrophy), other diseases (e.g., Alzheimer's disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostata, and vascular erectile dysfunction associated with aging and arteriosclerosis), inflammatory bowel disease (e.g., ulcerative colitis, Crohn's disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis.

Preferred disease and/or disorders are selected from the group consisting of ulcer, digital ulcer, diabetic gangrene, diabetic foot ulcer, pulmonary hypertension, pulmonary arterial hypertension, chronic thromboembolic pulmonary hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, and respiratory diseases.

Particularly preferred is pulmonary arterial hypertension (PAH). Particularly preferred is chronic thromboembolic pulmonary hypertension (CTEPH),

Calcium; {4-[(5,6-diphenylpyrazin-2-yl)(propan-2-yl)amino]butoxy}-acetate of formula (I) as prepared by the process described herein has a high purity. This is particularly important for the manufacturing of injectables, e.g. long acting injectables.

The calcium; {4-[(5,6-diphenylpyrazin-2-yl)(propan-2-yl)amino]butoxy}-acetate obtainable by the process as described herein, is therefore in particular suitable for the use in the treatment of the above-indicated diseases and/or disorders, preferably in the form of an intramuscular or subcutaneous injectable. Thereby, the injectable is a long-acting injectable (LAI). The term “long acting injectable” is used herein for an administration interval of one week to three months, or 1 week to two month, or 1 week to one month, or 1, 2, 3, 4, 5, 6, 7, 8, 9, 10, 11 or 12 weeks.

The present invention further concerns a method of treating a subject suffering from the above-indicated diseases and/or disorders, in particular PAH, said method comprising the administration of a therapeutically effective amount of calcium; {4-[(5,6-diphenylpyrazin-2-yl)(propan-2-yl)amino]butoxy}-acetate obtainable by the process as described herein. Preferably, the method comprises the administration of calcium; {4-[(5,6-diphenylpyrazin-2-yl)(propan-2-yl)amino]butoxy}-acetate obtainable by the process as described herein via intramuscular or subcutaneous injection.

The term “therapeutically effective amount” refers to amounts, or concentrations, of calcium; {4-[(5,6-diphenylpyrazin-2-yl)(propan-2-yl)amino]butoxy}-acetate that result in efficacious plasma levels for treating the indicated diseases, in particular PAH. For instance, a therapeutically effective amount may be 1 to 200 mg, for example 2 to 150 mg or 5 to 100 mg, and notably 25 mg to 100 mg of calcium; {4-[(5,6-diphenylpyrazin-2-yl)(propan-2-yl)amino]butoxy}acetate per month. With “efficacious plasma levels” it is meant those plasma levels of {4-[(5,6-diphenylpyrazin-2-yl)(propan-2-yl)amino]butoxy}acetic acid, that provide effective treatment or effective prevention of the indicated diseases and/or disorders, in particular PAH.

The term “subject” in particular relates to a human being.

All documents cited herein are incorporated by reference in their entirety.

The following examples are intended to illustrate the present invention and should not be construed as limiting the invention thereto.

Where present, all ranges are inclusive and combinable. That is, references to values stated in ranges include every value within that range. For example, a range defined as from 400 to 450 ppm includes 400 ppm and 450 ppm as independent embodiments.

Ranges of 400 to 450 ppm and 450 to 500 ppm may be combined to be a range of 400 to 500 ppm.

### EXAMPLES

Abbreviations (as used herein and in the description above):
ADME absorption, distribution, metabolism, and excretion
API Active Pharmaceutical Ingredient
aq. aqueous
h hour(s)
HPLC high performance liquid chromatography
IC Ca ion chromatography for calcium determination
IM intramuscular
INCI international nomenclature of cosmetic ingredients
INN international nonproprietary name
IP receptor prostacyclin receptor
ISO International Organization of Standardization
LAI long acting injectable
LC Assay liquid chromatography-quantitative analysis
min minute(s)
mM millimole
PAH Pulmonary Arterial Hypertension
CTEPH chronic thromboembolic pulmonary hypertension
pK pharmacokinetic
PVP polyvinylpyrrolidone
q.s. quantum satis (as much as is sufficient)
RH relative humidity
RT room temperature
SC subcutaneous
UPLC Ultra performance liquid chromatography
WFI water for injection
WHO World Health Organization
w/v weight per volume
w/w weight per weight
wt weight
XRPD X-ray powder diffraction

### X-Ray Powder Diffraction Analysis (XRPD)

### XRPD Method:

The XRPD diffractogram of Form 1 was collected on a PANalytical (Philips) X'PertPRO MPD diffractometer. The instrument was equipped with a Cu LFF X-ray tube.

The compound was spread on a zero background sample holder.

### Instrument Parameters

Generator voltage: 45 kV
Generator amperage: 40 mA
Geometry: Bragg-Brentano
Stage: spinner stage

### Measurement Conditions

Scan mode: continuous
Scan range: 3 to 50° 2θ
Step size: 0.02°/step
Counting time: 30 sec/step
Spinner revolution time: 1 sec
Radiation type: CuKa
Incident beam path Diffracted beam path
Program. divergence slit: 15 mm Long anti scatter shield: +
Soller slit: 0.04 rad Soller slit: 0.04 rad
Beam mask: 15 mm Ni filter: +
Anti scatter slit: 1° Detector: X'Celerator
Beam knife: +

The XRPD diffractogram of Form 2 was collected on a Bruker D8 diffractometer using Cu Kα radiation (40 kV, 40 mA) and a θ-2θ (theta-2theta) goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software for data collection and analysis was Diffrac Plus XRD Commander and Diffrac Plus EVA, respectively.

Samples were run under ambient conditions as flat plate specimens using powder. The sample was prepared on a polished, zero-background (510) silicon wafer by gently pressing onto the flat surface or packed into a cut cavity. The sample was rotated on its own plane.

### Details:

Angular range: 2 to 42° 2θ(theta)
Step size: 0.05° 2θ(theta)
Collection time: 0.5 s/step (total collection time: 6.40 min)

The XRPD diffractogram of Form 3 was collected on a PANalytical Empyrean diffractometer using Cu Kα radiation (40 kV, 40 mA) in transmission geometry. A 0.5° slit, 4 mm mask and 0.04 rad Soller slits with a focusing mirror were used on the incident beam. A PIXcel^{3D} detector, placed on the diffracted beam, was fitted with a receiving slit and 0.04 raf Soller slits. The software used for data collection was X'Pert Data Collector using X'Pert Operator Interface. The data were analysed and presented using Diffrac Plus EVA or HighScore Plus. Samples were prepared and analysed in a metal 96 well-plate in transmission mode. X-ray transparent film was used between the metal sheets on the metal well-plate and powders (approximately 1-2 mg) were used.

The scan mode for the metal plate used the gonio scan axis, whereas 2θ (theta) scan was utilised for the Millipore plate.

The details of the standard screening data collection method are:
Angular range: 2.5 to 32.0° 2θ(theta)
Step size: 0.0130° 2θ(theta)
Collection time: 12.75 s/step (total collection time: 2.07 min)

The XRPD diffractogram of Form 5 was collected on a Bruker D8 diffractometer (Bruker D8 Advance).

### XRPD Method:

Detector: LYNXEYE_XE_T (1D mode)
Open angle: 2.94°
Scan mode: Continuous PSD fast
Radiation: Cu/K-Alpha1 (gamma=1.5418 Angstrom)
X-ray generator power: 40 kV, 40 mA
Step size: 0.02°
Time per step: 0.12 second per step
Scan range: 3° to 40°
Primary beam path slits: Twin_Primary motorized slit 10.0 mm by sample length; SollerMount axial soller 2.5°
Secondary beam path slits: Detector OpticsMount soller slit 2.5°; Twin_Secondary motorized slit 5.2 mm
Sample rotation speed: 15 rpm

### Differential Scanning Calorimetry (DSC)

DSC data of Form 2 (and Form 3, respectively) were collected on a TA Instruments 02000 equipped with a 50-position auto-sampler. 1.5 mg of the material of Form 2 (1.7 mg in case of Form 3) was weight into a pin-holed aluminum pan, was heated at 10° C./min, from 25° C. to 250° C. A nitrogen purge at 50 mL/min was maintained over the sample. Peak temperatures are reported for melting points.

### Thermogravimetric Analysis (TGA)

TGA data of Form 2 (and Form 3, respectively) were collected on a TA Instruments Q500 equipped with a 16-position auto-sampler. Typically about 5-10 mg of a sample (7.7 mg in case of Form 2; 6.0 mg in case of Form 3) was loaded onto a pre-tared aluminum pan and was heated at 10° C./min, from 25° C. to 350° C. A nitrogen purge at 60 mL min-1 is maintained over the sample.

### Ion Chromatography

Calcium content was determined using ion chromatography. Sample preparation was done by weighing 10 mg of sample in a 50 mL flask. Approximately 25 mL MeOH:H₂O (50:50 v/v) was added whereafter a couple drops of conc. aq. HCl were added until a homogeneous solution was obtained (solution turns yellow). The sample was further diluted to volume using MeOH:H₂O (50:50 v/v). The resulting solution was diluted 2× by pipetting 10 mL into a 20 mL flask and diluting with the same dilution solvent. Analysis was performed using a Thermoscientific Dionex IC 5000+ ion chromatograph using a conductivity detector operating at 35° C. Separation was done on a Dionex IonPac CS12A (2×250 mm) analytical column, coupled to a Dionex IonPAc CG12A (2×250 mm) guard column using a column temperature of 30° C. An eluent generator cartridge was used to generate the eluent, methanesulfonic acid (MSA), which was delivered at a constant concentration of 20 mM during 15 minutes at a flow of 0.25 mL/min. Suppression was done using a Dionex CDRS 600 2 mm suppressor operating at 15 mA. Standard cation solutions containing Li, Na, K, Mg and Ca at 0.5, 1.0, 2.5, 5.0 and 10 ppm w/w were used for calibration. They were prepared starting from a commercially available 10 ppm IC cation standard solution (Merck) by dilution using MilliQ water. An injection volume of 10 uL was used for the analysis. The analytical error is 0.1%. Ca²⁺ results are provided in % w/w. The concentration of the analytes is automatically calculated by the Chromeleon software.

The Ca²⁺ content may be lower than the theoretical value of 4.5693% w/w in case the product contains water or other residual solvents, or in case there is a slight excess of selexipag metabolite.

### EXAMPLES

### Example 1: Preparation of Calcium; {4-[(5,6-diphenylpyrazin-2-yl)(propan-2-yl)amino]butoxy}acetate without Seeding

12 g (28.604 mmol) of {4-[(5,6-diphenylpyrazin-2-yl)(propan-2-yl)amino]butoxy}acetic acid were added to a two piece 400 ml reactor and 145.34 g of acetone/water (95/5% w/w) were added. Ramp stirring to a speed of 400 rpm was applied, and the reactor was heated to 50° C. at 1 K/min, and kept at that temperature for 30 min. Then, 15 vol % (4.2 ml) of Ca(OAc)₂×½H₂O dissolved in water (stock solution containing 2.51 g (15.012 mmol) Ca(OAc)₂×½H₂O in 26.66 g water)) were added over 30 min. The mixture was kept for 8 h. Then, the rest of the stock solution of Ca(OAc)₂ dissolved in water was added over 2 h. The mixture was stirred for 7.75 h, and the obtained solid was filtered off, washed with 24 g (2 g/g) acetone/water 80/20% w/w at 50° C. After drying at 50° C. under vacuum and N₂ purge, 12.46 g of crystalline calcium; {4-[(5,6-diphenylpyrazin-2-yl)(propan-2-yl)amino]-butoxy}acetate (99.3%) were obtained (crystalline Form 1). IC Ca²⁺ 4.41% w/w.

### Example 2: Preparation of Calcium; {4-[(5,6-diphenylpyrazin-2-yl)(propan-2-yl)amino]-butoxy}acetate with Seeding

### Phase 1: Dissolution

1.6 kg (3.814 mol) of {4-[(5,6-diphenylpyrazin-2-yl)(propan-2-yl)amino]butoxy}acetic acid were dissolved in 17.7776 kg of acetone/purified water 95/5% w/w. The reactor was heated to a reactor temperature of 50° C. at 1 K/min, then it was further stirred for 30 min.

### Phase 2: Polish Filtration

A polish filtration step was executed (CUNO filter of 0.5 micrometer), and the reactor was heated to a reactor temperature of 50° C. as fast as possible. The polish filter was washed with 0.8 kg acetone/purified water 95/5% w/w.

### Phase 3: Seeding and First Dosing of Ca(OAc)₂

Then, the solution was seeded with 16 g of crystals of calcium; {4-[(5,6-diphenylpyrazin-2-yl)(propan-2-yl)amino]butoxy}acetate (1% w/w based on 1.6 kg of starting material) and waited for 90 min. Then, 15 wt % of a solution of Ca(OAc)₂×½H₂O dissolved in purified water (stock solution containing 317.64 g (1.900 mol) Ca(OAc)₂×½H2O dissolved in 3.5552 kg water) was linearly dosed over 70 min to the mixture. The mixture was aged for 17 h.

### Phase 4: Second Dosing of Ca(OAc)₂

Then, the remaining 85 wtl % of the solution of Ca(OAc)₂ dissolved in purified water was linearly dosed over 271 min. The mixture was stirred for 19 h.

### Phase 5: Filtration and Drying

The obtained solid was filtered, and the filtrate washed with 3.2 kg acetone/purified water 80/20% w/w at 50° C. It was dried at 50° C., under vacuum and N₂ purge, and homogenized over a 2 mm sieve. 1.481 kg of calcium; {4-[(5,6-diphenylpyrazin-2-yl)(propan-2-yl)amino]butoxy}acetate (as defined in Formula (I)) (88.5% yield) were obtained.

### Optional Re-Slurry Phase 6:

In case the product of Phase 5 has a content of free {4-[(5,6-diphenylpyrazin-2-yl)(propan-2-yl)amino]butoxy}acetic acid higher than 2%, then a re-slurry step may take place as follows:

The product (1.481 kg) obtained above (after Phase 5) was re-slurried in 3.2 kg Ca(OAc)₂ dissolved in purified water (concentration=10 g/100 g). It was heated up to 50° C. with a rate of 1K/min, followed by stirring for 12 h. It was then cooled to 20° C. with a rate of 0.5K/min, and stirred for 4 h. It was filtered and washed with 2 times 16 kg of purified water, dried at 50° C. under vacuum and N₂ purge. After drying it was homogenized over a 2 mm sieve. Output: 1.377 kg (yield=93.0%,) IC Ca²⁺: 4.20% w/w

### Example 3: Preparation of Calcium; {4-[(5,6-diphenylpyrazin-2-yl)(propan-2-yl)amino]-butoxy}acetate Using Ca(OH)₂

59.9 g {4-[(5,6-Diphenylpyrazin-2-yl)(propan-2-yl)amino]butoxy}acetic acid, 10.6 g calcium hydroxide (1 molar eq.) and 1.5 L EtOH/water 50/50 vol % were added to a reactor. It was dissolved at 50° C. and stirred for 2 days. After 2 days, the solution was cooled to 20° C. The solid was isolated by vacuum filtration and air dried for 5 minutes, then dried at 50 C° C. in a vacuum oven for 16 h. 64 g (102%) of calcium; {4-[(5,6-diphenylpyrazin-2-yl)(propan-2-yl)amino]-butoxy}acetate was isolated (still containing residual Ca(OH)₂). IC Ca²⁺: 7.57% w/w

### Example 4: Preparation of Amorphous Form of Calcium; {4-[(5,6-diphenylpyrazin-2-yl)(propan-2-yl)amino]butoxy}acetate

Calcium; {4-[(5,6-diphenylpyrazin-2-yl)(propan-2-yl)amino]butoxy}acetate (30 g) (obtained from a batch similar to example 3) was heated to 210° C. in an oven for 20 min until the sample melted. The molten material was then rapidly cooled to −18° C. to give a glass.

The anhydrous form remains physically stable (amorphous) after 7 days storage at 25° C./97% RH and 40° C./75% RH condition.

### Example 5: Preparation of Form 2 of Calcium; {4-[(5,6-diphenylpyrazin-2-yl)(propan-2-yl)amino]butoxy}acetate

To amorphous calcium; {4-[(5,6-diphenylpyrazin-2-yl)(propan-2-yl)amino]butoxy}acetate (30 mg) was added 0.6 mL of methanol, and the sample was slurried at 60° C. in a platform shaker incubator for 6 days, and the obtained solid was isolated as Form 2.

Form 2 is an anhydrous form which melts at 175.6° C. with a heat of fusion of 46 J/g. It contains an additional endotherm at 122.9° C. (5 J/g). TGA analysis shows weigh losses attributed to loss of water of 0.3% between RT-100° C., and 1.0% between 100-200° C.

Form 2 is slightly hygroscopic (shows a reversible 2% change in mass between 0 and 90% RH), and physically stable after 7 days storage at 25° C./97% RH and 40° C./75% RH conditions.

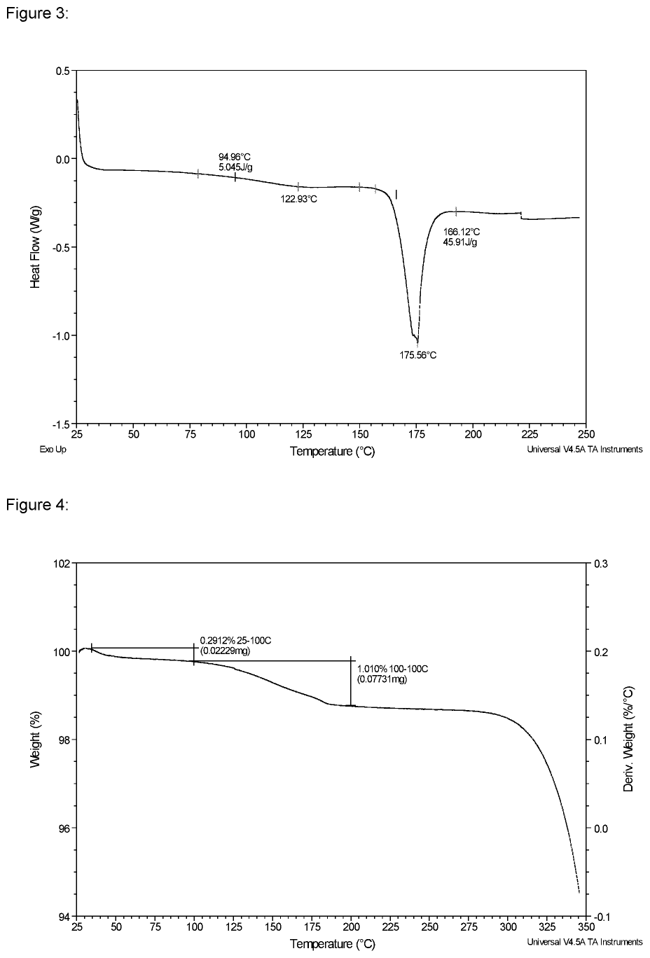

X-ray pattern, DCS and TGA are shown in FIGS. 2, 3 and 4.

### Example 6: Preparation of Form 3 of Calcium; {4-[(5,6-diphenylpyrazin-2-yl)(propan-2-yl)amino]butoxy}acetate

To amorphous calcium; {4-[(5,6-diphenylpyrazin-2-yl)(propan-2-yl)amino]butoxy}acetate (300 mg) was added 6 mL of anisole, and the sample was stirred at 60° C., 500 rpm for 8 days. The resulting white suspension was separated by filtration and dried in a vacuum oven at RT overnight, to obtain Form 3.

Form 3 was shown to be a group of isostructural solvates isolated from toluene and anisole, i.e. it is also obtained from the same procedure using toluene.

TGA analysis shows a weight loss of 10.8% (RT-190° C.) and 1.1% between 190-270° C. (total mass loss 10.9%, equal to 0.5 mol eq. anisole). DSC shows broad endothermic signal with a maximum at 164.9° C. (87 J/g) due to melting/collapsing of solvated form. An additional endothermic signal is observed at 196.2° C. (3 J/g) during further heating and corresponds to the melting of Form 1.

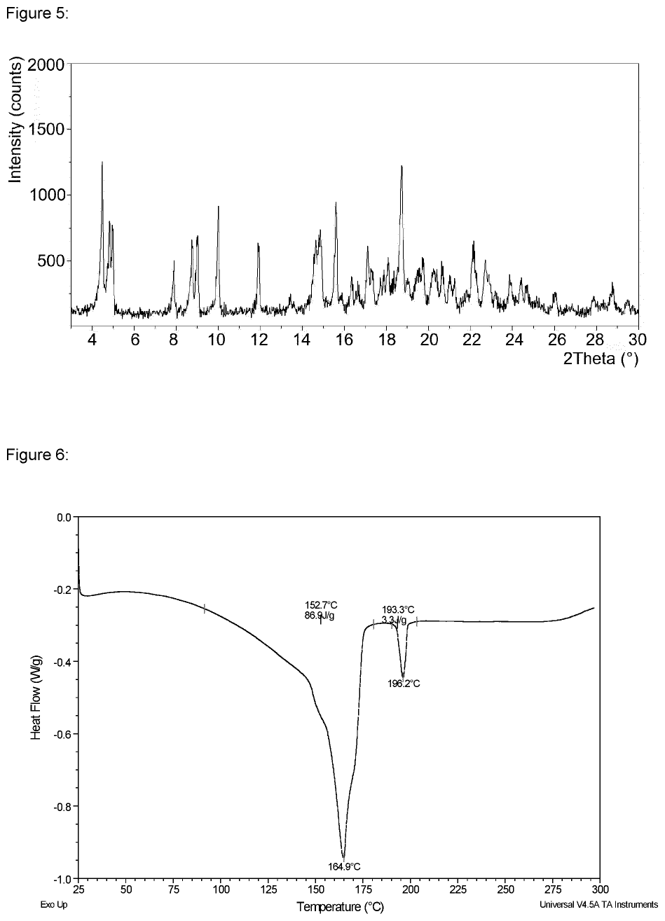

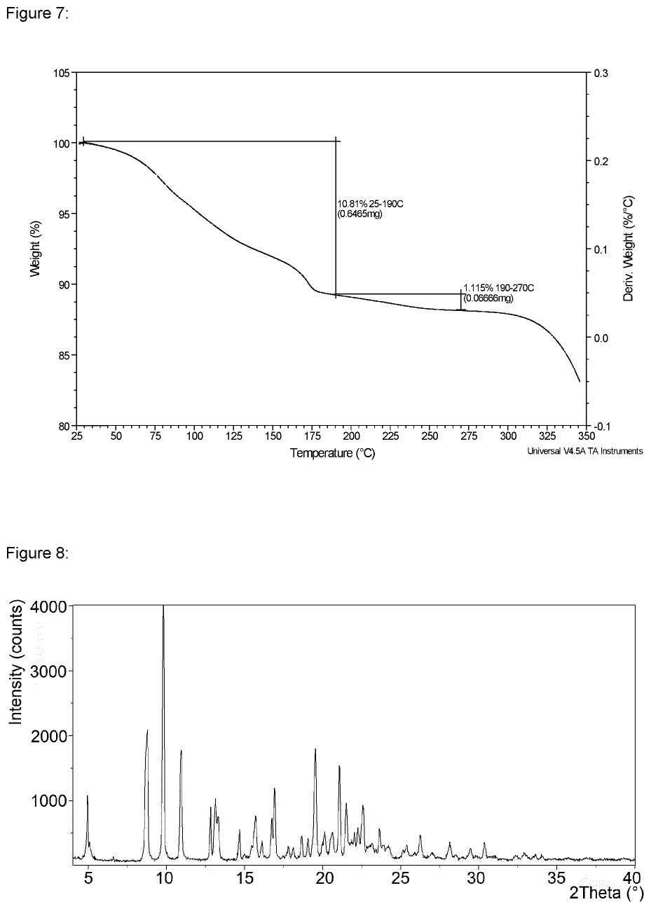

X-ray pattern, DSC and TGA of Form 3 are shown in FIGS. 5, 6 and 7.

### Example 7: Preparation of Form 5 of Calcium; {4-[(5,6-diphenylpyrazin-2-yl)(propan-2-yl)amino]butoxy}acetate

Form 5 is the dehydration product of Form 1 and was obtained from a variable temperature XRD experiment performed on Form 1. At a temperature of 190° C. (RT to 190° C. and hold 2 min; 190° C. to 25° C. and hold 2 min), Form 1 converted to Form 5; when temperature is back to 25° C., Form 5 converts back to Form 1. This result also suggests that hydrate Form 1 exhibits reversible dehydration-hydration behaviour.

The X-ray pattern of Form 5 is shown in FIG. 8.

### Example 8: Feasibility pK Rat Study

An initial pK rat study was conducted to demonstrate the LAI potential of an aqueous micro-suspension of calcium; {4-[(5,6-diphenylpyrazin-2-yl)(propan-2-yl)amino]butoxy}acetate. For this study, aqueous micro-suspensions of Selexipag, {4-[(5,6-diphenylpyrazin-2-yl)(propan-2-yl)amino]butoxy}acetic acid and calcium; {4-[(5,6-diphenylpyrazin-2-yl)(propan-2-yl)amino]butoxy}acetate were prepared. An overview of the design of the study can be found in Table 1.

TABLE 1
pK rat study design (ADME) to demonstrate LAI feasibility

| | | | API eq. | Particle | | |
|---|---|---|---|---|---|---|
| | | | concen- | size | | Dose |
| | | | tration | (Dv50, | Injection | (mg/ |
| Group | Formulation | API | (mg/mL) | μm) | route | kg) |
| F | PVP K17 | Selexipag | 125 | 6.6 | IM | 50 |
| | Citrate | | | | | |
| | buffer | | | | | |
| | pH 5 | | | | | |
| G | PVP K17 | Ca-salt of | 125 | 3.5 | IM | 50 |
| | Citrate | selexipag | | | | |
| | buffer | metabolite | | | | |
| | pH 8 | | | | | |
| H | PVP K17 | Selexipag | 125 | 4.3 | IM | 50 |
| | Citrate | metabolite | | | | |
| | buffer | | | | | |
| | pH 5 | | | | | |

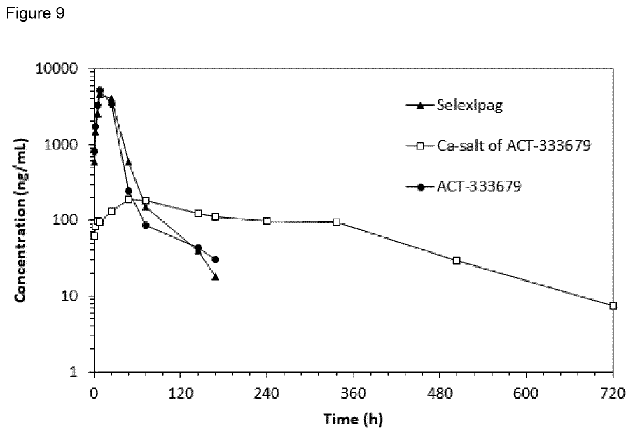

Release profiles and mean AUC of the different formulations are depicted in FIG. 9.

As shown in FIG. 9, the study group dosed with the Ca-salt of ACT-333679 exhibits significant lower plasma concentrations compared to both other groups, which demonstrates a long-acting release profile up to 336 hours (i.e. 14 days) and the AUC increases up until 720 hours. Selexipag and its metabolite (group F and H) did not demonstrate a long-acting release profile because of their high solubility and dissolution rate.

### Example 9: Preparation of Calcium; {4-[(5,6-diphenylpyrazin-2-yl)(propan-2-yl)amino]-butoxy}acetate Using Other Calcium Sources with High Water Solubility

6.9 g of {4-[(5,6-diphenylpyrazin-2-yl)(propan-2-yl)amino]butoxy}acetic acid were dissolved in 100 g of acetone/purified water 95/5% w/w at 50° C. The first calcium source dissolved in water (0.5 mol/mol) was added to the mixture at a rate of 0.1 mL/min until there was a ratio of acetone/water of 70/30% w/w. The mixture was stirred for 4 hours. The solid was isolated at 50° C. and washed with 2 g/g acetone/water 70/30% w/w. The product was dried at 50° C. Results are shown in Table 2.

TABLE 2

| | | | LC | Yield | XRD |
|---|---|---|---|---|---|
| Ca source | IC Ca | LC Assay | Impurities | (% F/F) | pattern |
| calcium | 4.31% | 99.5% | none | 92.8% | Form 1 |
| propionate | | | | | |
| calcium | 4.31% | 101.6% | none | 86.1% | Form 1 |
| formate | | | | | |
| calcium | 22.0% | 0.40% | none | 15.3% | * |
| hypophosphite | | | | | |
| calcium | 8.17% | 63.7% | none | 23.6% | Form 1 |
| pyruvate | | | | | |
| calcium | 5.73% | 78.7% | none | 63.1% | Form 1 |
| L-lactate | | | | | |
| hydrate | | | | | |

* indicates that the form of the material was not determined because of the low yield

### Example 10: Preparation of Calcium; {4-[(5,6-diphenylpyrazin-2-yl)(propan-2-yl)amino]-butoxy}acetate Using Other Calcium Sources with High Water Solubility

6.9 g of {4-[(5,6-diphenylpyrazin-2-yl)(propan-2-yl)amino]butoxy}acetic acid were dissolved in 100 g of acetone/purified water 80/20% w/w at 50° C. The first calcium source dissolved in water (0.5 mol/mol) was added to the mixture at a rate of 0.1 mL/min until there was a ratio of acetone/water of 55/45% w/w. The mixture was stirred for 4 hours. The solid was isolated at 50° C. and washed with 2 g/g acetone/water 55/45% w/w. The product was dried at 50° C. Results are shown in Table 3.

TABLE 3

| | | | LC | Yield | XRD |
|---|---|---|---|---|---|
| Ca source | IC Ca | LC Assay | Impurities | (% F/F) | pattern |
| calcium | 1.50% | 96.6% | none | 81.6% | mixture of |
| pyruvate | | | | | Form 1 and |
| | | | | | free acid |
| | | | | | Form 2 |
| calcium | 3.68% | 92.5% | none | 86.1% | mixture of |
| L-lactate | | | | | Form 1 and |
| hydrate | | | | | free acid |
| | | | | | Form 2 |
| L-glyceric acid | 3.05% | 82.2% | none | 92.5% | mixture of |
| hemicalcium | | | | | Form 1 and |
| salt | | | | | free acid |
| monohydrate | | | | | Form 2 |

### Example 11: Preparation of Calcium; {4-[(5,6-diphenylpyrazin-2-yl)(propan-2-yl)amino]-butoxy}acetate Using Other Calcium Sources with Medium Water Solubility in Water

1 g of {4-[(5,6-diphenylpyrazin-2-yl)(propan-2-yl)amino]butoxy}acetic acid were dissolved in 100 g of acetone/purified water 80/20% w/w at 50° C. The first calcium source dissolved in water (0.5 mol/mol) was added to the mixture over 4 hours until there was a ratio of acetone/water of 55/45% w/w. The mixture was stirred for 17-22 hours. The solid was isolated at 50° C. and washed with 2 g/g acetone/water 55/45% w/w. The product was dried at 50° C. Results are shown in Table 4.

TABLE 4

| | | | LC | Yield | XRD |
|---|---|---|---|---|---|
| Ca source | IC Ca | LC Assay | Impurities | (% F/F) | pattern |
| edetic acid | | | | 2.9% | * |
| calcium | | | | | |
| disodium salt | | | | | |
| calcium | 8.35% | 0.32% | none | 37.8% | n.d. |
| D-gluconate | | | | | |
| as monohydrate | | | | | |
| D-glyceric | | | | 1.4% | * |
| acid calcium | | | | | |
| salt dihydrate | | | | | |

* = the form of the material was not determined because of the low yield
n.d. = the form of the material was not determined

### Example 12: Preparation of Calcium; {4-[(5,6-diphenylpyrazin-2-yl)(propan-2-yl)amino]-butoxy}acetate Using Other Calcium Sources with Low Water Solubility

1 g of {4-[(5,6-diphenylpyrazin-2-yl)(propan-2-yl)amino]butoxy}acetic acid in 100 g of acetone/purified water 70/30% w/w and 0.5 mol/mol first calcium source was stirred for 5 days at 50° C. The solid was isolated at 50° C. and washed with 2 g/g acetone/water 70/30% w/w. The product was dried at 50° C. Results are shown in Table 5.

TABLE 5

| | | | LC | Yield | XRD |
|---|---|---|---|---|---|
| Ca source | IC Ca | LC Assay | Impurities | (% F/F) | pattern |
| calcium | 4.17% | 96.1% | none | 79.9% | Form 1 |
| pantothenate | | | | | |
| calcium citrate | 20.7% | 0.17 | none | 59.8% | n.d. |
| as tribasic | | | | | |
| tetrahydrate | | | | | |
| calcium | | | | 9.1% | * |
| oxalate | | | | | |
| monohydrate | | | | | |
| calcium | 21.2% | 0.21% | none | 18.2% | n.d. |
| malate | | | | | |
| calcium | 13.2% | 0.2% | 1.31% | 22.0% | n.d. |
| threonate | | | | | |

* = the form of the material was not determined because of the low yield
n.d. = the form of the material was not determined

1. A process for the manufacturing of
calcium; {4-[(5,6-diphenylpyrazin-2-yl)(propan-2-yl)amino]butoxy}acetate of formula (I), or a pharmaceutically acceptable hydrate or solvate thereof: 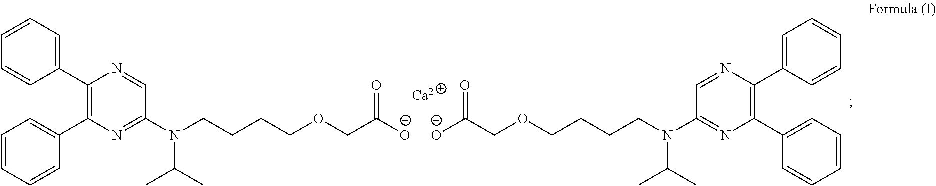 [SMILES: CC(C)N(CCCCOCC(=O)[O-])c1cnc(-c2ccccc2)c(-c2ccccc2)n1 ; CC(C)N(CCCCOCC(=O)[O-])c1cnc(-c2ccccc2)c(-c2ccccc2)n1]
comprising the steps of:
mixing {4-[(5,6-diphenylpyrazin-2-yl)(propan-2-yl)amino]butoxy}acetic acid and a first calcium source with a solvent (a) to obtain a mixture;
heating or maintaining the mixture at a temperature in the range of 20° C. to 85° C.;
isolating the obtained solid product; and
optionally re-slurrying the isolated solid product in a solution of a second calcium source in solvent (b) at a temperature in the range of 20° C. to 85° C.

2. The process of claim 1, wherein the mixing step comprises:
mixing {4-[(5,6-diphenylpyrazin-2-yl)(propan-2-yl)amino]butoxy}acetic acid and solvent (a) to obtain a mixture; and
heating or maintaining the mixture at a temperature in the range of 20° C. to 85° C. prior to the addition of the first calcium source.

3. The process of claim 1, wherein the first calcium source is dissolved in solvent (b) to obtain solution (b) prior to the addition of solution (b) to the mixture of {4-[(5,6-diphenylpyrazin-2-yl)(propan-2-yl)amino]butoxy}acetic acid and solvent (a).

4. The process of claim 1, wherein the steps comprise:
(1) dissolving {4-[(5,6-diphenylpyrazin-2-yl)(propan-2-yl)amino]butoxy}acetic acid in a solvent (a) to obtain solution (a);
(2) heating solution (a) to a temperature in the range of 20° C. to 85° C.;
(3) dissolving a first calcium source in solvent (b) to obtain solution (b);
(4) dosing solution (b) to solution (a);
(5) isolating the obtained solid product; and
(6) optionally re-slurrying the product of step (5) in a solution of a second calcium source in solvent (b) at a temperature in the range of 20° C. to 85° C.

5. The process of claim 1, wherein the first calcium source is added in an amount of 0.4 mol to 1 mol per mol
{4-[(5,6-diphenylpyrazin-2-yl)(propan-2-yl)amino]butoxy}acetic acid, or from 0.4 to 0.8 mol per mol {4-[(5,6-diphenylpyrazin-2-yl)(propan-2-yl)amino]butoxy}acetic acid, or from 0.4 to 0.6 mol per mol
{4-[(5,6-diphenylpyrazin-2-yl)(propan-2-yl)amino]butoxy}acetic acid, or from 0.45 to 0.6 mol per mol {4-[(5,6-diphenylpyrazin-2-yl)(propan-2-yl)amino]butoxy}acetic acid, or from 0.45 to 0.55 mol per mol
{4-[(5,6-diphenylpyrazin-2-yl)(propan-2-yl)amino]butoxy}acetic acid or from 0.5 to 0.6 mol per mol {4-[(5,6-diphenylpyrazin-2-yl)(propan-2-yl)amino]butoxy}acetic acid, or from 0.5 to 0.55 mol per mol
{4-[(5,6-diphenylpyrazin-2-yl)(propan-2-yl)amino]butoxy}acetic acid.

6. The process of claim 1, wherein the first calcium source is added in two or more dosages.

7. The process of claim 1, wherein solvent (a) is an organic solvent or an organic solvent mixed with water.

8. The process of claim 7, wherein the organic solvent in solvent (a) is selected from the group consisting of acetone, tetrahydrofuran (THF), acetonitrile, MEK (methyl ethyl ketone), DMSO, DMF, 1,4-dioxane, pyridine, dimethylacetamide (DMA), methyl acetate (MeOAc), methanol, ethanol, propanol (1-propanol, or 2-propanol), and butanol (1-butanol, 2-butanol, 2-methylpropan-1-ol, or 2-methylpropanol).

9. The process of claim 4, wherein solution (a) is subjected to a filtering step.

10. The process of claim 1, wherein solvent (b) is selected from water or a mixture of water and an organic solvent.

11. The process of claim 1,
wherein seed crystals of
calcium; {4-[(5,6-diphenylpyrazin-2-yl)(propan-2-yl)amino]butoxy}acetate of formula (I) are added to solution (a) or the mixture in an amount of up to 25% w/w in respect of amount of {4-[(5,6-diphenylpyrazin-2-yl)(propan-2-yl)amino]butoxy}acetic acid used as starting material.

12. The process of claim 11, wherein the seed crystals of calcium; {4-[(5,6-diphenylpyrazin-2-yl)(propan-2-yl)amino]butoxy}acetate of formula (I) have an X-ray powder diffraction pattern with at least five peaks, or at least seven peaks, or at least nine peaks having angle of refraction 2θ (2theta) values selected from: 5.1°, 5.4°, 8.8°, 9.9°, 11.4°, 13.4°, 13.8°, 16.3°, 19.7°, 20.9°, 21.4°, 22.9°, 25.1°, wherein the Xray powder diffraction diagram is obtained by using Cu Kα radiation, wherein the accuracy of the 2θ (2theta) values is in the range of 2θ+/−0.2° (2theta+/−0.20).

13. The process of claim 1, wherein the first, second, or both calcium sources are selected from Ca(OAc)₂, calcium propionate, calcium formate, and calcium pantothenate.

14. The process of claim 13, wherein the first calcium source and the optional second calcium source is Ca(OAc)₂.

15. The product obtained by the process of claim 1.

16-23. (canceled)
